(19) United States
(12) Patent Application Publication
(10) Pub. No.: US 2009/0254148 A1
(43) Pub. Date: Oct. 8, 2009

(54) Telemetrically Controllable System for Treatment of Nervous Sytem Injury

(76) Inventors: Richard B. Borgens, Delphi, IN (US); Pedro Irazoqui, Lafayette, IN (US)

Correspondence Address: David B Quick;Ice Miller, One American Square Suite 3100, Indianapolis, IN, 46282-0200, US

(21) Appl. No.: 12/083,794

(22) PCT Filed: Oct. 20, 2006
(86) PCT No.: PCT/US2006/040820

§ 371 Date: Apr. 18, 2008

Related U.S. Application Data
(60) Provisional application No. 60/729,247, filed on Oct. 21, 2005.

(51) Int. Cl. A61N 1/02

(52) U.S. Cl. 607/50

(57) ABSTRACT

An apparatus (500) for stimulating axon growth of the nerve cells in the spinal cord of mammals to stimulate regeneration of the nerve cells in the spinal cord. Such an apparatus includes a variable current DC stimulus generator (420), and data transfer circuitry (410) in communication with the DC stimulus generator (420) and an external module (430), the data transfer circuitry (410) operable to transmit signals between the DC stimulus generator (420) and the external module (430). The DC stimulus generator (420) and data transfer circuitry (410) may be within a biocompatible container (510).

### BACKGROUND

Injury to the spinal cord or central nervous system can be one of the most devastating and disabling injuries possible. Depending upon the severity of the injury, paralysis of varying degrees can result. Paraplegia and quadriplegia often result from severe injury to the spinal cord. The resulting effect on the sufferer, be it man or animal, is severe. The sufferer can be reduced to a state of near immobility or worse. For humans, the mental trauma induced by such severe physical disability can be even more devastating than the physical disability itself.

When the spinal cord of a mammal is injured, connections between nerves in the spinal cord are broken. The injured portion of the spinal cord is termed a “lesion.” Such lesions block the flow of nerve impulses for the nerve tracts affected by the lesion with resulting impairment to both sensory and motor function.

To restore the lost sensory and motor functions, the affected motor and sensory axons of the injured nerves must regenerate, that is, grow back. Unfortunately, any spontaneous regeneration of injured nerves in the central nervous system of mammals has been found to occur, if at all, only within a very short period immediately after the injury occurs. After this short period expires, such nerves have not been found to regenerate further spontaneously.

Studies have shown, however, that the application of a DC electrical field across a lesion in the spinal cord of mammals, can promote axon growth, and the axons will grow back around the lesion. Since the spinal cord is rarely severed completely when injured, the axons need not actually grow across the lesion but can circumnavigate the lesion through remaining spinal cord parenchyma.

For optimal results in a human patient, a uniform electrical field of a desired strength is imposed over about 10 cm to 20 cm of damaged spinal cord for a beneficial clinical outcome. Ideally, this uniform field is imposed across the entire cross section of the spinal cord over this longitudinal extent, because of the general segregation of descending (motor) tracts to the ventral (anterior) cord, and the segregation of important (largely sensory) tracts to the posterior (dorsal) spinal cord. In paraplegic canines, this electrical field has been directly measured (Richard B. Borgens, James P. Toombs, Andrew R. Blight, Michael E. McGinnis, Michael S. Bauer, William R. Widmer, and James R. Cook Jr., Effects of Applied Electric Fields on Clinical Cases of Complete Paraplegia in Dogs, J. Restorative Neurology and Neurosci., 1993, pp. 5:305-322). In man however, the cross sectional area of the spinal cord is approximately two to four times that of the small to medium sized dogs treated in clinical trials, and actual invasive measurement of the imposed electrical fields in response is not feasible on human patients.

Based on the responses of human paraplegics and quadriplegics to prior art therapies involving the application of an oscillating DC electrical field across a lesion in the spinal cord using three pairs of electrodes, it appears that the dorsal (posterior) location of three pairs of electrodes did not produce a uniform field over the entire unit area of the patient's spinal cord. This was revealed by the domination of sensory recovery in these patients (<thirty fold over historical controls) compared to motor recovery (˜twofold greater than historical controls) using the ASIA scoring system. Thus, the voltage gradient was highest nearest to the actual placement of two pairs of electrodes on either side (two tethered to the right and left lateral facets) and the third pair sutured to the paravertebral muscle and fascia of the dorsal (posterior) facet-rostra and caudal of the spinal cord lesion (Shapiro, et al., Oscillating Field Stimulation for Complete Spinal Cord Injury in Humans: a Phase 1 Trial, Journal of Neurosurg. Spine 2, 2005, pp. 3-10).

It would be desirable to provide a device to generate a stronger DC electrical field across the spinal cord lesion of a human in order to facilitate the creation of a uniform electrical field over the affected area. It would be further desirable to provide a method for implanting electrodes that facilitates the creation of a uniform electrical field over the affected area of the injured spinal cord.

### SUMMARY

According to at least one aspect of the disclosure, an apparatus for stimulating axon growth of the nerve cells in the spinal cord of mammals to stimulate regeneration of the nerve cells in the spinal cord comprises a variable current DC stimulus generator, polarity reversing lo circuitry, and data transfer circuitry. Such a variable current DC stimulus generator has first and second groups of oppositely polarized output electrodes, wherein one group of electrodes comprises at least three electrodes acting as a cathode of the generator, and the other group of output electrodes comprises at least three electrodes acting as an anode of the generator. Such polarity reversing circuitry is configured to reverse the polarity of the DC stimulus each time a predetermined period of time elapses, wherein each time the polarity of the DC stimulus is reversed the output electrodes which comprised the cathode before the polarity reversal comprises the anode after the polarity reversal and the output electrodes which comprised the anode before the polarity reversal comprises the cathode after the polarity reversal. Such data transfer circuitry is in communication with the DC stimulus generator, and is operable to transmit signals to and from the DC stimulus generator.

According to at least one aspect of the disclosure, an apparatus for stimulating axon growth of the nerve cells in the spinal cord of mammals comprises a DC stimulus controller that controls the duty cycle of a DC stimulus generator to provide an on-cycle wherein the generator provides a DC output and an off cycle wherein the generator does not provide a DC output, the duty cycle being generated during each polarity reversal.

According to at least one aspect of the disclosure, an apparatus for stimulating axon growth of the nerve cells in the spinal cord of mammals comprises a DC stimulus controller that controls the amplitude of the DC stimulus generator to provide an on-cycle wherein the generator provides a DC output and an off cycle wherein the generator does not provide a DC output, the duty cycle being generated during each polarity reversal.

According to at least one aspect of the disclosure, an apparatus for stimulating axon growth of the nerve cells in the spinal cord of mammals comprises a DC stimulus controller that controls the frequency of the DC stimulus generator to provide an on-cycle wherein the generator provides a DC output and an off cycle wherein the generator does not provide a DC output, the duty cycle being generated during each polarity reversal.

According to at least one aspect of the disclosure, an apparatus for stimulating axon growth of the nerve cells in the spinal cord of mammals comprising a variable current DC stimulus generator, first and second groups of electrodes, and data transfer circuitry are each components configured to be implanted in the body of a patient suffering nerve cell damage.

According to at least one aspect of the disclosure, an apparatus for stimulating axon growth of the nerve cells in the spinal cord of mammals comprises an external controller for controlling the output of a DC stimulus generator. Such an external controller may be communicatively coupled with data transfer circuitry in an apparatus for stimulating axon growth of the nerve cells in the spinal cord of mammals. Such an external controller and data transfer circuitry may be capable of bi-directional communication, which may be accomplished via radio frequency transmission.

According to at least one aspect of the disclosure, data transfer circuitry may comprise at least one low-pass filter, at least one transceiver, at least one voltage controlled oscillator, and at least one antenna.

According to at least one aspect of the disclosure, an apparatus for stimulating axon growth of the nerve cells in the spinal cord of mammals to stimulate regeneration of the nerve cells in the spinal cord comprises a variable current DC stimulus generator, polarity reversing circuitry, data transfer circuitry, and at least one has sensor, wherein at least one sensor is capable of monitoring the electrical environment surrounding the apparatus. Such data transfer circuitry may be capable of telemetering information about the electrical environment surrounding the apparatus to an external device. Such an external device may, in response to the information about the electrical environment surrounding the apparatus, generate configuration information and transmit the configuration information to the data transfer circuitry, where the configuration information comprises parameters for controlling the output of the DC stimulus generator. Such an apparatus may comprise first and second groups of electrodes, wherein at least one electrode is configured as at least one sensor.

According to at least one aspect of the disclosure, an apparatus for stimulating axon growth of the nerve cells in the spinal cord of mammals to stimulate regeneration of the nerve cells in the spinal cord comprises a variable current DC stimulus generator, polarity reversing circuitry, data transfer circuitry, and at least one has sensor, wherein at least one sensor is capable of monitoring the biological environment surrounding the apparatus. Such data transfer circuitry may be capable of telemetering information about the biological environment surrounding the apparatus to an external device. Such an external device may, in response to the information about the biological environment surrounding the apparatus, generate configuration information and transmit the configuration information to the data transfer circuitry, where the configuration information comprises parameters for controlling the output of the DC stimulus generator. Such an apparatus may comprise first and second groups of electrodes, wherein at least one electrode is configured as at least one sensor.

Additional features and advantages of the invention will become apparent to those skilled in the art upon consideration of the following detailed description of a preferred embodiment exemplifying the best mode of carrying out the invention as presently perceived.

### BRIEF DESCRIPTION OF THE DRAWINGS

The features and advantages of this disclosure, and the methods of obtaining them, will be more apparent and better understood by reference to the following descriptions of disclosed embodiments, taken in conjunction with the accompanying drawings, wherein:

FIG. 1 shows a graph that portrays the effect of an applied steady DC field over time on the growth of cathodal and anodal facing axons;

FIG. 2 shows a graph that portrays the effect of an applied oscillating field over time on the growth of cathodal and anodal facing axons;

FIG. 3A shows a first portion of a schematic of a circuit for generating an oscillating electrical field for stimulating nerve regeneration;

FIG. 3B shows a second portion of a schematic of a circuit for generating an oscillating electrical field for stimulating nerve regeneration;

FIG. 4A shows a block diagram of a neural injury treatment device;

FIG. 4B shows a schematic of a second circuit for generating an oscillating electrical field for stimulating nerve regeneration;

FIG. 5 shows a schematic of a current source of the circuit of FIG. 4B;

FIG. 6 shows a schematic of a voltage controlled oscillator of the circuit of FIG. 4B;

FIG. 7 shows a schematic of an electromagnetic power coupling portion of the circuit of FIG. 4B; and

FIG. 8A shows a first portion of a schematic of a biphasic pulse generator that may serve as the pulse generator of the circuit of FIG. 4B;

FIG. 8B shows a second portion of a schematic of a biphasic pulse generator that may serve as the pulse generator of the circuit of FIG. 4B;

FIG. 9 is a wave diagram of a triphasic pulse;

FIG. 10 is a block diagram of a triphasic pulse generator that may serve as the pulse generator of the circuit of FIG. 4B;

FIG. 11 shows a graph that portrays the effect of an applied pulse wave modulated oscillating field over time on the growth of cathodal and anodal facing axons.

### DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present invention includes any alterations and modifications to the illustrated embodiments and includes further applications of the principles of the disclosure as would normally occur to one skilled in the art to which this invention pertains.

The application of an oscillating DC electrical field across a lesion in the spinal cord of a mammal can stimulate axon growth in both directions, i.e., caudally and rostrally. That is, growth of caudally facing axons will be promoted as will growth of rostrally facing axons. The DC electrical field is a stimulus which is first applied in one direction for a predetermined period of time and then applied in the opposite direction for the predetermined period of time. The polarity of the DC stimulus is reversed after each predetermined period of time.

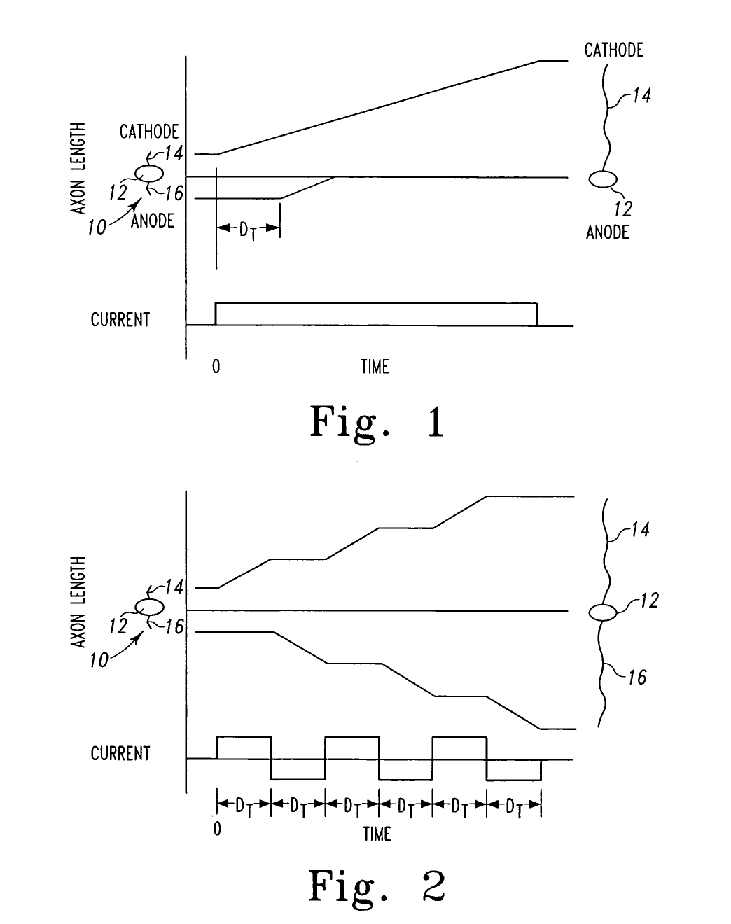

FIGS. 1 and 2 show the effects on axon growth by an applied steady state DC electrical field (FIG. 1) and by an applied oscillating electrical field (FIG. 2). Referring to FIG. 1, a nerve cell 10 is shown at the left-hand side of FIG. 1 having a cell body or soma 12 from which an axon 14 extends upwardly and an axon 16 extends downwardly. At time 0, a DC stimulus having a first polarity is applied to the nerve cell 10 such that axon 14 will be extending toward the cathode or negative pole of a DC stimulus signal and axon 16 will be extending toward the anode or positive pole of the DC stimulus. Axon 14 begins to grow almost immediately. However, after a period of time, i.e., the “die back period” (D_{T}), significant reabsorption of axon 16 into the cell body 12 begins and eventually axon 16 is completely reabsorbed into cell body 12. At the right-hand side of FIG. 1, for illustration purposes, nerve cell 10 is shown wherein axon 14 has grown substantially longer but axon 16 has been reabsorbed into cell body 12.

In FIG. 2, the same reference numbers will be used to identify the elements of FIG. 2 which correspond to elements of FIG. 1. Nerve cell 10 is shown at the left-hand side of FIG. 2 having a cell body 12, an upwardly extending axon 14 and a downwardly extending axon 16. At time 0, a DC stimulus having a first polarity is applied to nerve cell 10 such that axon 14 is extending toward the cathode and axon 16 is extending toward the anode of the DC stimulus. After a predetermined period of time, the polarity of the DC stimulus is reversed. Axon 14 will now be extending toward the anode and axon 16 will be extending toward the cathode of the DC stimulus. The predetermined period of time is selected to be less than the die back period (D_{T}) of the anodal facing axon. Significant die back of anodal facing axons begins to occur about one hour after the DC stimulus is applied. Therefore, the predetermined period should not exceed one hour. As shown in FIG. 2, an oscillating DC field stimulates growth of the axons facing both directions. This is due to the fact that growth of cathodal facing axons is stimulated almost immediately after the DC stimulus is applied but die back of the anodal facing axons does not become significant until after the die back period elapses. Since the polarity of the DC stimulus is switched before the die back period elapses, growth of axons in both directions is stimulated with the result that axons 14, 16 of nerve cell 12 both grow significantly longer as shown, for example, at the right-hand side of FIG. 2.

In accordance with the present disclosure, the nerves in the central nervous system of a mammal are stimulated to regenerate by applying an oscillating electrical field to the central nervous system. The oscillating electrical field is a DC stimulus which is first applied in one direction, i.e. having a first polarity, for a predetermined period of time, and then applied in the opposite direction, i.e. having a second polarity opposite to the first polarity, for the predetermined period of time. In other words, the polarity of the DC stimulus is reversed after each predetermined period of time. The predetermined period of time is selected to be less than the die back period of anodal facing axons, but long enough to stimulate growth of cathodal facing axons. This pre-determined period of time will also be termed the “polarity reversal period” of the oscillating electrical field. In one disclosed embodiment, the polarity reversal period is between about thirty seconds and about sixty minutes. According to at least one embodiment of the present disclosure, there may be a period between each polarity reversal period where no voltage potential stimulus is applied (an “off cycle”). According to at least one embodiment of the present disclosure, two or more consecutive polarity reversal periods may be followed by an off cycle.

Prior art technology generates a DC voltage of about 600 μV/mm that is imposed along the long axis of an injured spinal cord, and the polarity of the voltage is reversed about every 15 minutes to induce regrowth and reconnection of both ascending (towards the brain) and descending (towards the body) white matter tracts (containing only nerve fibers). In the prior art technology, therapy usually had to be discontinued within 14 to 16 weeks because of the capacity of the voltage source. Moreover, the waveform, or shape of the electrical signal based on electrical-field magnitude, the duty cycle, and the continuous DC, could not be altered.

While prior art application of DC voltages have proved useful, patients who do not respond optimally may benefit from second or third regimens of therapy. This can be carried out at the discretion of the clinician with other unrelated therapies to affect an improved clinical outcome. For example, use of injections of soluble “neurotrophic” factors (BDNF, Interleukins, Inosine, etc) that can be administered for short times clinically may boost the growth response of nerve fibers experiencing DC voltages, and may extend the time post injury during which DC voltage applications are effective.

Additionally, new research has revealed that this prior art method may not produce an optimum stimulation to achieve optimum results in clinical recovery. For example, in one new method of therapy, pulsatile DC fields in duce nerve regeneration. Where the DC Voltage of one polarity is “chopped” (turned on and off rapidly), no loss in its functional properties appears to occur. Intermittent DC fields also guide and induce growth. In this case, an “off” time relatively shorter than the “on time” increases the growth response to a level equal to or greater than that achieved by a steady DC field. Moreover, a substantially decreased power consumption may be possible by this mode of stimulation. For example, during a fifteen minute long imposition of the DC field on the spinal cord injury of a single polarity before duty cycle reversal, a forty-five second “on” time followed by a 15 second “off” time provides a saving in power consumption equivalent to at least about twenty-five percent of that used in the nominal duty cycle.

While DC voltages induce growth towards the cathode in physiological milieu, they also may significantly reduce or eliminate retrograde degeneration of nerve fibers that have undergone secondary axotomy and have broken in two. The proximal segment (that in connection with the nerve cell body) usually survives while the distal segment (that disenfranchised from the cell body) usually dies and is lost, a process that in mammals known as Wallerian Degeneration. This is the fate of most mechanically damaged fibers following any form of nerve injury in the mammal. As distally negative extracellular voltage reduces the endogenous calcium current entering the cut end of the fiber, the concentration of calcium ions in the terminal axoplasm, and thus the “dieback” of the process is dependent on this. This distance produced by dieback, which can typically vary between tenths of a millimeter to several centimeters of the terminal nerve fiber, must be “made up” to permit extension of the fiber past the region of local injury.

Another approach to therapy is to use a steady DC stimulus application in the early stages of the injury (e.g., 96 hours up to about post-injury), when most secondary axotomy occurs, reducing the “dieback” of nerve fibers, followed by a change in the waveform carried out by the clinician to improve regeneration and functional outcome, for example, back to a pusitile or time varying steady DC Field. In short, varying the field parameters may allow a more direct attack on retrograde degeneration of nerve fibers producing a better overall growth response dependent on the extent of linear growth. The devices disclosed herein may implement these new approaches to therapy.

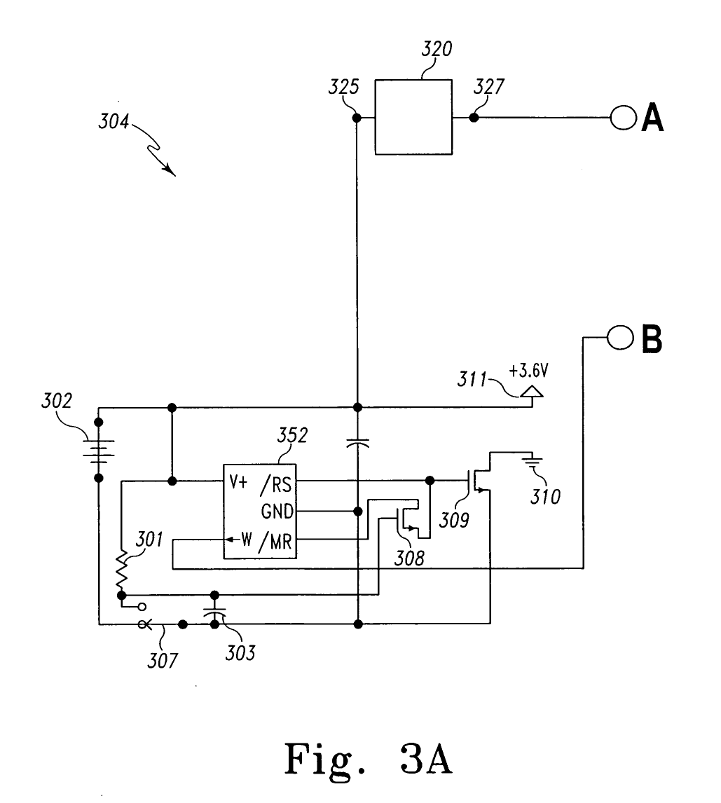

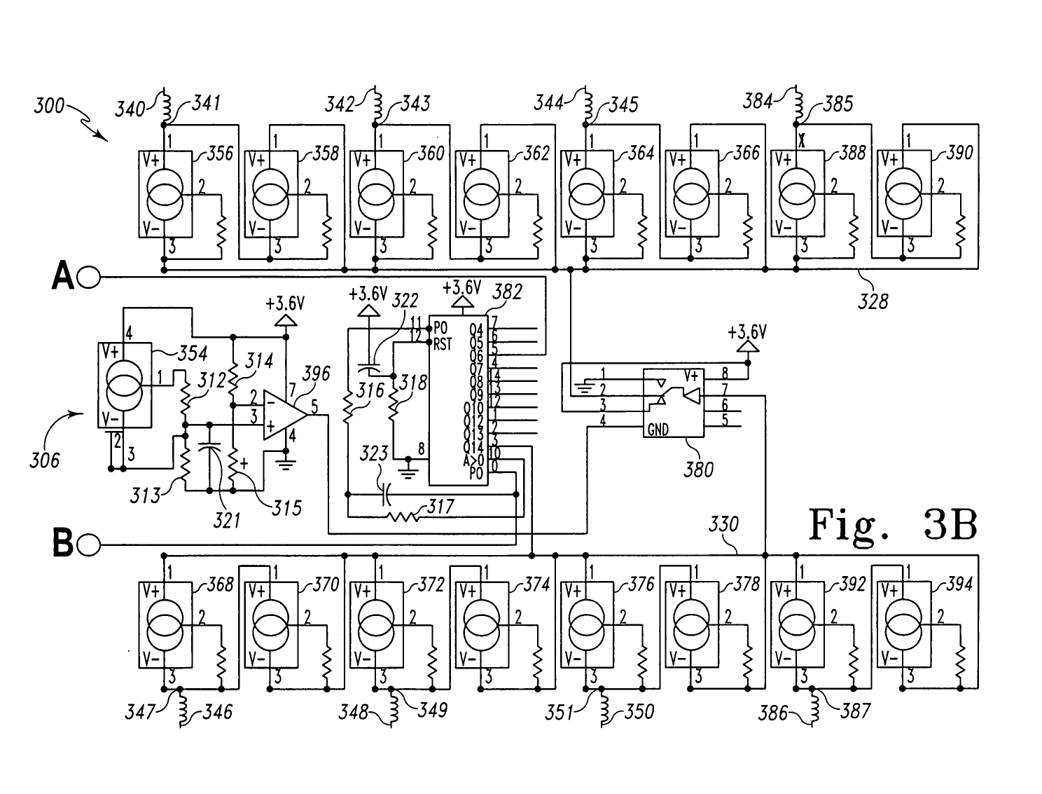

FIGS. 3A and 3B (which together make up FIG. 3) show a schematic of a circuit 300 for generating an oscillating electrical field for stimulating nerve regeneration. The circuit 300 comprises electronic components electrically interconnected as shown in FIG. 3. Conventional symbols are used to denote the components. The circuit 300 as shown in FIG. 3 comprises electrodes 340, 342, 344, 346, 348, 350, 384 and 386; processor supervisory circuit 352; adjustable current sources 354, 356, 358, 360, 362, 364, 366, 368, 370, 372, 374, 376, 378, 388, 390, 392 and 394; switch 380; and timer 382. The circuit 300 as shown in FIG. 3 also comprises an optional beacon circuit 320, which is electrically interconnected between nodes 325 and 327. Electrodes 340, 342, 344 and 384 comprise Electrode Group A. Electrodes 346, 348, 350 and 386 comprise Electrode Group B.

Electrode 340 is coupled to the output terminal 341 of the back-to-back adjustable current sources 356 and 358 which constitute a portion of the DC stimulus generator. Electrode 342 is coupled to the output terminal 343 of the back-to-back adjustable current sources 360 and 362 which constitute a portion of the DC stimulus generator. Electrode 344 is coupled to the output terminal 345 of the back-to-back adjustable current sources 364 and 366 which constitute a portion of the DC stimulus generator. Electrode 384 is coupled to the output terminal 385 of back-to-back adjustable current sources 388 and 390 which constitute a portion of the DC stimulus generator. Electrodes 340,342,344 and 384 comprise Electrode Group A and thus output terminals 341, 343, 345 and 385 constitute one group of output terminals.

Electrode 346 is coupled to the output terminal 347 of the back-to-back adjustable current sources 368 and 370 which constitute a portion of the DC stimulus generator. Electrode 348 is coupled to the output terminal 349 of the back-to-back adjustable current sources 372 and 374 which constitute a portion of the DC stimulus generator. Electrode 350 is coupled to the output terminal 351 of the back-to-back adjustable current sources 376 and 378 which constitute a portion of the DC stimulus generator. Electrode 386 is coupled to the output terminal 387 of back-to-back adjustable current sources 392 and 394 which constitute a portion of the DC stimulus generator. Electrodes 346, 348, 350 and 386 comprise Electrode Group B and thus output terminals 347, 349, 351 and 387 constitute another group of output terminals.

Circuit 300 includes a power supply and supervisory section 304, and a secondary watchdog section 306. The power supply and supervisory section 304 produces a 3.6 volt supply for powering the remaining devices of circuit 300, including secondary watchdog section 306 and the optional beacon circuit 320 and the main oscillator of timer 382. Additionally, the power supply and supervisory section 306 supervises the oscillator circuitry of the timer 382 to determine if there is failure of the oscillator circuit.

The power supply and supervisory circuit 304 includes a battery 302, processor supervisor circuit 352, a resistor 301, a first capacitor 303, a second capacitor 305, a switch 307, a first transistor 308, and a second transistor 309 configured as shown in FIG. 3 to provide a 3.6 volt potential between a ground terminal 310 and a positive voltage terminal 311 for so long as the oscillator circuitry of the timer 382 is operating within desired parameters as explained in greater detail below. In one illustrated embodiment, the battery 302 may be a 3.6v Tadiran TL-5903 battery although other charge storage devices, including, but not limited to, rechargeable charge storage devices, e.g. charge storage device 429, may be used within the scope of the disclosure.

In one illustrated embodiment, the switch 307 may be an HSR-502RT reed switch available from Hermetic Switch, Inc., Chickasha, Okla. However, other switches may be used within the scope of the disclosure. The HSR-502 reed switch is a single pole-double throw (SPDT) switch enclosed in a glass capsule.

In one illustrated embodiment, transistors 308 and 309 may be BSS138 transistors available from Fairchild Semiconductor Corporation, South Portland, Me., although other transistors and appropriate components can be used within the scope of the disclosure. In one illustrated embodiment, the transistors 308, 309 are N-Channel Logic Level Enhancement Mode Field Effect Transistors. The values of the resistor 301 and capacitors 303, 305 are chosen as required to meet design parameters. In the illustrated embodiment, resistor 301 is a 1 Mohm resistor and capacitors 303, 305 are 0.047 microfarad capacitors.

The processor supervisor circuit 352 receives a clock pulse signal from the oscillator section of timer 382. In one illustrated embodiment, the processor supervisor circuit 352 is a TPS 3823 Processor supervisor circuit with watchdog timer input (W) and Manual Reset Input (/MR) available from Texas Instruments, Dallas Tex. The illustrated processor supervisor circuit 352 includes a Power-On Reset Generator With Fixed Delay Time of 200 ms. The illustrated processor supervisor circuit 352 provides circuit initialization and timing supervision for the timer 382. During power-on, /RESET (/RS) is asserted when supply voltage (V+) becomes higher than 1.1 V. Thereafter, the supply voltage supervisor monitors the supply voltage and keeps /RESET active as long as the supply voltage remains below the threshold voltage. An internal timer delays the return of the output to the inactive state (high) to ensure proper system reset. The delay time, td, starts after supply voltage has risen above the threshold voltage. When the supply voltage drops below the threshold voltage, the output becomes active (low) again. The illustrated processor supervisory circuit 352 has a fixed-sense threshold voltage set by an internal voltage divider. The illustrated processor supervisor circuit 352 incorporates a manual reset input, (/MR). A low level at the manual reset input (/MR) causes /RESET to become active. The illustrated processor supervisor circuit 352 includes a high-level output at /RESET (/RS).

The arrangement illustrated in FIG. 3 is configured so that when a low level is received on the /RESET pin of the processor supervisor circuit 352, the gate of the transistor 308 receives no current effectively shutting down transistor 309. When transistor 309 is shut down, the power supply is effectively shut down causing the remaining components of the circuit 300 to be without power. Once transistor 309 is shut down, transistor 308 asserts a low signal on the /MR pin of the supervisor circuit 352 effectively locking down the circuit until the power is cycled utilizing switch 307. This configuration of timer 382, supervisory circuit 352 and transistors 308, 309 acts as a failsafe device to shut down the oscillating field circuit whenever there is an apparent failure of the oscillator of the timer 382 so that the axons facing anodes will not be subjected to a disadvantageously oriented electrical field beyond the beginning of the die back period.

The illustrated processor supervisor circuit 352 includes watchdog timer that is periodically triggered by a positive or negative transition at the watchdog timer input (W). The watchdog timer receives the clock pulse from the timer 382 of the secondary watchdog section 306. When the supervising system fails to retrigger the watchdog circuit within the time-out interval, tₜₒᵤₜ, /RESET becomes active which, as described above shuts down transistor 309 and causes transistor 308 to assert a low signal on the /MR pin of the process supervisor circuit. This event also locks down and removes power from all of the other components of the circuit 300 (except battery 302) until power is cycled via switch 307.

The positive terminal of the battery 302 is electrically connected to the supply voltage input (V+) of the processor supervisory circuit 352, one terminal of resistor 301, the positive electrode of the second capacitor 305 and to the positive output terminal 311. The second terminal of the resistor 301 is electrically connected to a node electrically connected to one terminal of the switch 307, the positive electrode of the first capacitor 303 and the gate of the reset transistor 308 of the above described power-on/reset delay network. The second terminal of the switch 307 is electrically connected to the negative terminal of the battery 302. The pole of the switch 307 is electrically connected to a node electrically connected to the negative electrode of the first capacitor 303, the ground pin (GND) of the processor supervisor circuit 352, the negative electrode of the second capacitor 305 and the source of the second transistor 309. The gate of the second transistor 309 is coupled to a node coupled to the /RESET pin (/RS) and the source of the first transistor 308. The drain of the second transistor 309 is coupled to the ground terminal 310. The drain of the first transistor is coupled to the manual reset pin (/MR) of the processor supervisor circuit 352. The watchdog timer input (W) of the processor supervisor circuit 352 is coupled to the PO pin of the timer 382.

The secondary watchdog section 306 includes adjustable current supply 354, switch 380, op amp 396, resistors 312-315 and capacitors 321. While the illustrated secondary watchdog section 306 is configured in accordance with the schematic shown in FIG. 3, it is within the scope of the disclosure for the secondary watchdog section 306 to be configured using other or additional components or for the section to be implemented on a single or multiple integrated circuits or a portion of a single or multiple integrated circuits implementing circuit 300.

In one illustrated embodiment, op amp 396 is an Analog Devices OP90GS Precision, Low Voltage Micropower Operational Amplifier, available from One Technology Way, Norwood, Mass. Other operational amplifiers or amplifier circuitry may be utilized within the scope of the disclosure.

In one illustrated embodiment, the switch 380 is a MAX4544CSA Low-Voltage, Single-Supply Dual SPDT Analog Switch available from Maxim Integrated Products, Sunnyvale, Calif. The MAX4544 is a dual analog switch designed to operate from a single voltage supply, which because of its low power consumption (5 μW) is particularly well adapted for battery-powered equipment. The disclosed switch 380 offers low leakage currents (100 pA max) and fast switching speeds (tON=150 ns max, tOFF=100 ns max). The MAX4544 switch 380 is a single pole/double-throw (SPDT) device.

In one illustrated embodiment, the timer 382 is a CD4060B type CMOS 14-stage ripple-carry binary counter/divider and oscillator, available from Texas Instruments, Dallas, Tex. The illustrated CD4060B timer 382 consists of an oscillator section and 14 ripple-carry binary counter stages. A RESET input is provided which resets the counter to the all-0's state and disables the oscillator. A high level on the RESET line accomplishes the reset function. All counter stages are master-slave flip-flops. The state of the counter is advanced one step in binary order on the negative transition of PI (and PO). All inputs and outputs are fully buffered. Schmitt trigger action on the input-pulse line permits unlimited input-pulse rise and fall times.

In the illustrated embodiment, the watchdog timer input to the processor supervisor circuit 352 is coupled to the PO output of the timer 382 to provide a pulsed clock signal to indicate proper operation of the timer 382 which controls the polarity reversal period. Absence of this signal causes the supervisor circuit 352 to shut down power to the entire system. The /PO pin of the timer 382 is coupled through resistors 316 and 317 to the PI pin of the timer 382. The positive electrode of capacitor 323 is coupled to a node coupling the terminals of resistors 316 and 317, while the negative electrode of the capacitor 323 is coupled to a node coupled to the PO pin of the timer 382 thereby forming a free running oscillator. The period of the free-running oscillator is determined by the values of the resistors 316 and 317 and the capacitor 323. In the illustrated embodiment, the resistors 316 and 317 each have a resistance of 1 Mohm and the capacitor 323 has a 0.047 micro-farad capacitance so that the oscillator runs at a frequency to generate the desired reversal period. The values of the resistors 316 and 317 and capacitor 323 can be varied to obtain reversal periods of different values within the scope of the disclosure.

The Q6 pin of the counter of the timer 382 is coupled to node 327 to provide a pulse to activate the optional beacon circuit 320. The Q14 pin of the timer 382 is coupled to a group B node 330, i.e. a node providing power to the adjustable current sources 368, 370, 372, 374, 376, 378, 392 and 394 driving the Group B electrodes 346, 348, 350 and 386. The reset pin of the timer 382 is coupled to a node that is coupled through the capacitor 322 to the positive voltage terminal 311 and coupled through resistor 318 to a node coupled to both the ground terminal 310 and the ground pin of the timer 382. The power supply pin of the timer 382 is coupled to the positive voltage terminal 311.

The adjustable current source 354 of the secondary watchdog section 306 has its positive supply pin (V+) coupled to a node coupled to the positive voltage terminal 311. This adjustable current source 354 provides a reference current that is utilized by op amp 396 to generate a signal to turn off the output power when the voltage drops below a specified value (illustratively 2.8V). In the illustrated embodiment, the adjustable current source 354 was selected to generate a second reference voltage instead of selecting a zenor diode to avoid the power loss associated with zenor diodes when utilized as reference voltage generators. The output power is interrupted in the illustrated circuit 300 by adjustable current source 354 and op amp 396 cooperating to lift the ground of switch 380 to interrupt current outflow to the group A electrodes.

The negative pin (V−) of the adjustable current source 354 is coupled to the central node of a first voltage divider formed by resistors 312 and 313. The central node of the first voltage divider is coupled through the resistor 313 to the ground terminal 310 and is also coupled through a node to the non-inverting input of op amp 396. The capacitor 321 is in parallel with the resistor 313 between the central node of the first voltage divider and the ground terminal 310. The resistors 314 and 315 form a second voltage divider having a central node coupled to the inverting input of the op amp 396. The second voltage divider is coupled between the positive voltage terminal 311 and the ground terminal 310. The positive voltage terminal 311 is also coupled to the voltage supply pin of the op amp 396 and the ground terminal 310 is coupled to the ground pin of the op amp 396. The output of the op amp is coupled to the Ground-Negative Supply Input pin of the switch 380.

The Positive Supply Voltage Input pin of the switch 380 is coupled to the positive voltage terminal 311. The Ground-Negative Supply Input pin of the switch 380 is coupled to the output of the op amp 396. The Normally Open pin of the switch 380 is coupled to the ground terminal 310. The Common pin of the switch 380 is coupled to the Group A node, i.e. the node 328 for providing the power to the adjustable current supplies 356, 358, 360, 362, 364, 366, 388 and 390 powering the Group A electrodes 340, 342, 344 and 384. The Normally Closed pin of the switch 380 is coupled to the positive voltage terminal 311. The Digital Control Input pin of the switch 380 is coupled to the Group B node 330 which, as mentioned above, is also coupled to the Q14 pin of the timer 382. Thus, the timer 382 is configured to cause the Group A electrodes and Group B electrodes to switch between anodes and cathodes to generate a waveform such as that shown in FIG. 2.

In operation, a device comprising circuit 300 is implanted into an injured mammal shortly after the time of central nervous system injury. The device comprising circuit 300 remains implanted for a period of time post-injury. For example, the device comprising circuit 300 may remain implanted for up to fourteen weeks or longer in humans.

Power may be applied to the device comprising circuit 300 for a period of time while the device is implanted. When power is applied, the circuit 300 generates an oscillating electrical field between at Electrode Group A and Electrode Group B. That is, the circuit 300 may generate a current DC stimulus the polarity of which is reversed periodically after the expiration of a predetermined period of time. The predetermined period of time may be determined by the operation of the timer 382. Electrode Group A and Electrode Group B alternately comprise cathode and anode terminals, depending upon the polarity of the DC stimulus.

The voltage between Electrode Group A and Electrode Group B is selected so as to provide sufficient field strength in the section of the spinal cord in which nerve regeneration is to be stimulated. A field strength of 200 μV/mm in the spinal cord will stimulate regeneration. The current needed to achieve this field strength is determined by the geometry of the animal in which a device comprising the circuit 300 is used.

Illustratively, electrodes 340, 342, 344, 346, 348, 350, 384, and 386 may comprise silastic insulated platinum electrodes. Electrode Groups A and B are implanted on opposite sides of a lesion in the spinal cord. It is sufficient to implant Electrode Groups A and B in a laminectomy adjacent the spinal cord but not actually in the spinal cord. Further, moving the anode from within the laminectomy to a site on the muscle dorsal to the same area results in only about a ten percent drop in field strength as does the converse of moving the cathode to a more superficial position while leaving the anode in the laminectomy.

Significant recovery of sensory function (ascending functions) has resulted from prior art technology, however, motor recovery has not been as robust. This has been documented in treatment of clinical paraplegia in dog and paraplegia/quadriplegia in man. The major sensory columns in mammals (Dorsal Coolum /Medial Lenmiscus system) are located bilaterally in the dorsal or posterior spinal cord, while long tract motor columns are located in the anterior (ventral) cord, as are the location of the major, bilaterally located, alpha motorneuron plexus in the upper (pectoral) and lower (lumbosacral) intumescences of the spinal cord.

Applicants have also found that the field strength within the spinal cord at the site of the lesion depends upon the location of the current delivery electrodes. The convergence of current to an electrode produces high current density and hence higher field strength near each electrode. The closer one electrode is to the lesion site, the less critical is the placement on the other to maintain high field strengths. However, as a current delivery electrode approaches the lesion, current direction becomes less uniform.

At a lesion exactly half-way between two electrodes placed on the midline, the current will all be oriented along the long axis of the subject animal. As one of the electrodes is moved closer to the lesion, there will be a larger vertical (dorsal-ventrical) component of the current at the lesion (assuming that the electrodes remain a few millimeters dorsal to the target tissue). As a compromise between uniform current direction and maximum field strength, applicants have chosen to position the electrodes two vertebral segments on either side of the lesion in their spinal cord studies. In the guinea pig studies applicants have conducted, it appears that the critical distance to be within one convergence zone of an electrode (that area in which the current convergence to the electrode so dominates the field strength that the position of the other electrode is relatively inconsequential) is approximately 1 cm. Therefore, by placing one electrode within 1 cm of the lesion, the position of the other becomes relatively inconsequential and becomes a matter of convenience. It should be noted, however, that the electrodes can be located further from the lesion. If they are, the field strength of the electrical field at the lesion for a given magnitude of current will be reduced. Therefore, the magnitude of the current would have to be increased to yield the same electrical field strength at the lesion.

For optimal results in a human patient, uniform electrical field of the desired strength is imposed over about 10 cm to 20 cm of damaged spinal cord for a beneficial clinical outcome. Ideally, this uniform field is imposed across the entire cross section of the spinal cord over this longitudinal extent, because of the general segregation of descending (motor) tracts to the ventral (anterior) cord, and the segregation of important (largely sensory) tracts to the posterior (dorsal) spinal cord. This uniform electrical field of the desired strength may be generated by placing two pairs of electrodes, for example electrodes 340, 346, 342, and 348, on either side (two tethered to the right and left lateral facets) and a third pair, for example electrodes 344 and 350, sutured to the paravertebral muscle and fascia of the dorsal (posterior) facet-rostra and caudal of the spinal cord lesion. Additionally, a fourth pair of electrodes, for example 384 and 386, are sutured to paravertebral musculature at the extreme mediolateral/ventral (anterior) vertebral column. The placement of this fourth pair of electrodes 384 and 386 should alleviate the reduction of the voltage gradient imposed over motor columns in the anterior (ventral) spinal cord.

Once inside a patient, it is difficult to verify the operation of a device comprising circuit 300. Visible verification is virtually impossible while the device is within a patient. Operation of the device within the patient could be determined by attaching an electrocardiogram (EKG) system to the patient and waiting to observe a small transient on the EKG record associated with the reversal of the electrical field imposed over the spinal cord, but this is a time consuming procedure.

Optional beacon circuit 320 can be used with circuit 300 to enable rapid verification of device operation. Beacon circuit 320 can be any circuit that enables visible and/or audible verification of device operation. Beacon circuit 320 also can transmit data regarding device operation, such as, for example, using RF telemetry. In an embodiment, a small LED “beacon” is inserted into circuit 300. A periodic visible burst of light such as, for example, every 7 seconds, reveals nominal unit operation prior to implantation.

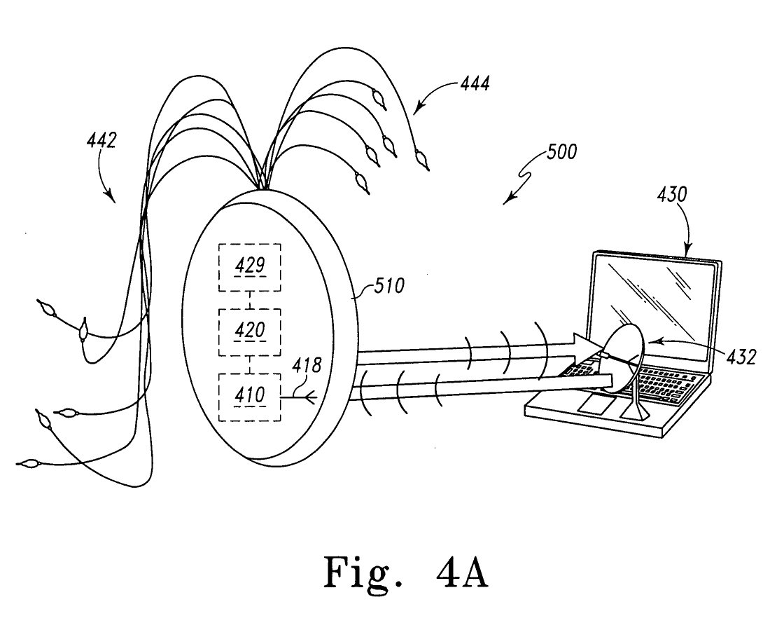

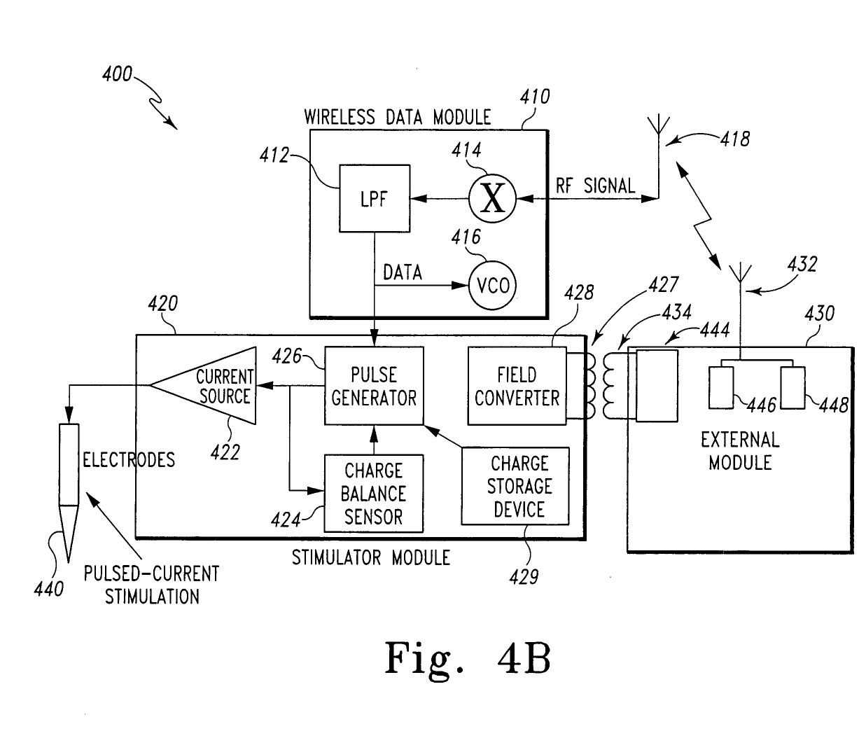

FIG. 4A shows a neural injury treatment device 500. The neural injury treatment device 500 includes a skin 510. The skin 510 may comprise a ceramic and/or titanium or other bio-compatible material, making the neural injury treatment device 500, in theory, surgically implantable for the life of the patient. The skin 510 may provide a container for the electronics of the neural injury treatment device 500. In one embodiment, the skin 510 may comprise medically approved ceramic available from the Sigma-Aldrich Corporation. In another embodiment, the skin 510 may comprise a Titanium cases. In yet another embodiment skin 510 may comprise a titanium portion, a bio-compatible material portion and a ceramic portion.

One advantage of a skin 510 comprising ceramic is that lifetime implantable ceramic cases provide the ability to mold the container into a desired shape. Additionally, because ceramic is transparent to electromagnetic radiation, it may be desirable to fabricate at least a “window” of the skin 510 from ceramic in order to facilitate the transmission of electromagnetic waves carrying power and data. The ceramic material used to fabricate the skin 510 may be obtained as a powder to facilitate the custom molding of shapes. For example, one useful shape for the skin 510 may be to mold the container into the form of an intervertebral disc or vertebral facet for certain applications in the spinal/orthopaedic management of fracture/dislocation associated spinal cord injuries. Because ceramic is transparent to electromagnetic waves, such a skin 510 facilitates the functionality of telemetry, antennae 418, fail-safe off, and other capabilities associated with telemetry.

The neural injury treatment device 500 may also include a wireless data module 410, a stimulator module 420, a charge storage device 429, a first electrode group 442 (Electrode Group A) and a second electrode group 444 (Electrode Group B). The first and second electrode groups 442 and 444 may comprise silastic insulated platinum electrodes, similar to the electrodes described above. The neural injury treatment device 500 may also include an external module 430. The external module 430 may include data acquisition 446, device programming 448, and inductive power-coupling hardware 444 configured to interface with the wireless data module 410 and the stimulator module 420, as shown, for example, in FIG. 4B.

FIG. 4B shows a schematic of the circuit 400 for generating an oscillating electrical field for stimulating nerve regeneration. The circuit 400 provides a means to treat spinal cord injury, as well as other nerve cell injuries. The circuit 400 facilitates these treatments by providing imposed gradients of DC voltage between about 200 to about 900 μV/mm. These voltage gradients may induce functional regeneration and reconnection of mechanically injured neural axons in vertebrates.

The circuit 400 may include the wireless data module 410, the stimulator module 420, the external module 430, and the electrodes 440. The wireless data module 410 may include a low-pass filter 412, a transceiver 414, a voltage controlled oscillator 416, and antenna 418. The low-pass filter 412 may be an active amplifier with low-frequency cutoff. The low-pass filter 412 may also include or be comprised of on-chip or off-chip passive resistive and capacitive devices. The transceiver 414 may be a mixer. The voltage controlled oscillator 416 may be a cross-coupled high-frequency oscillator. The antenna 418 may be a planar microstip antenna or a monolithic microwave integrated-circuit (MMIC) radiating structure integrated with or bonded to an application specific integrated circuit. The components of the circuit 400 may be CMOS or BiCMOS.

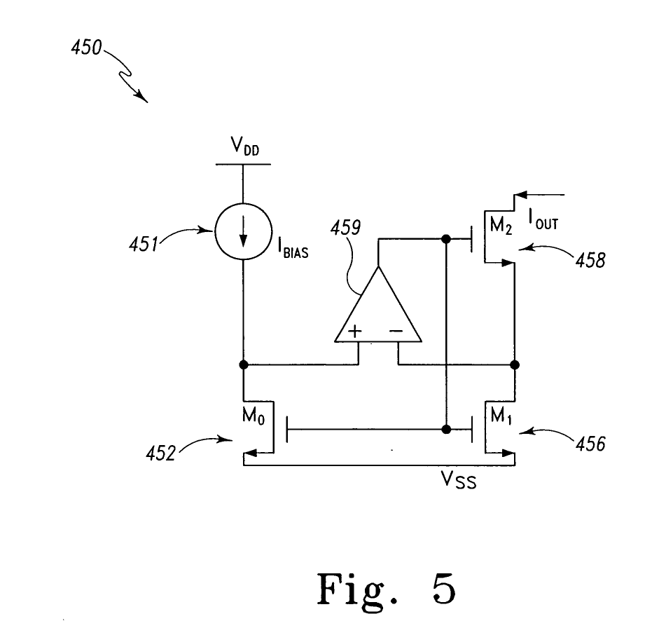

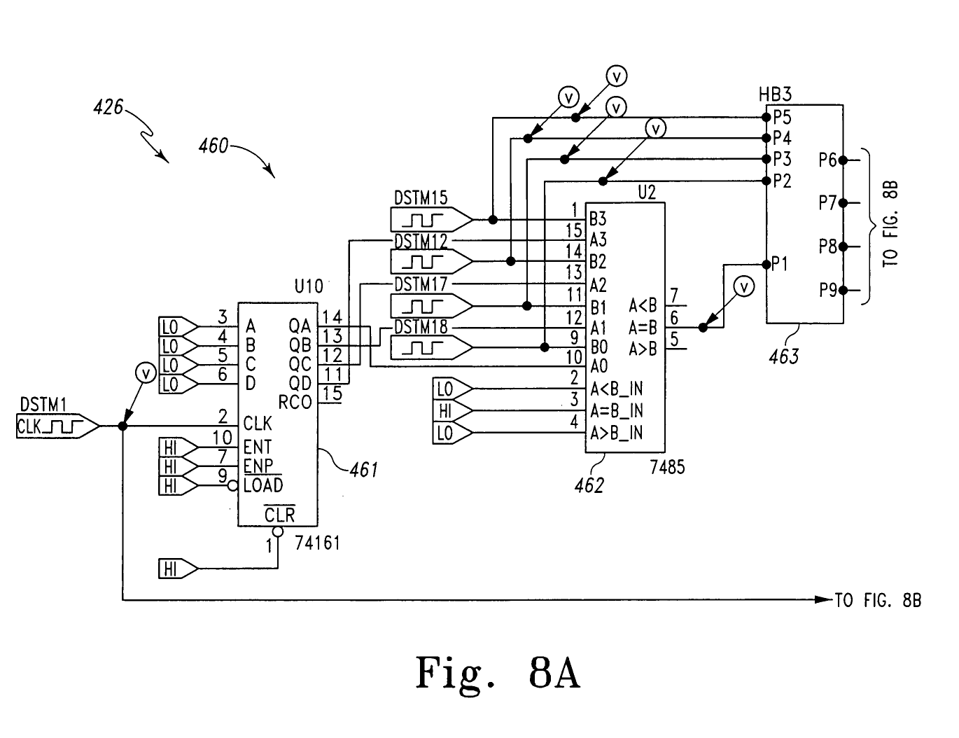

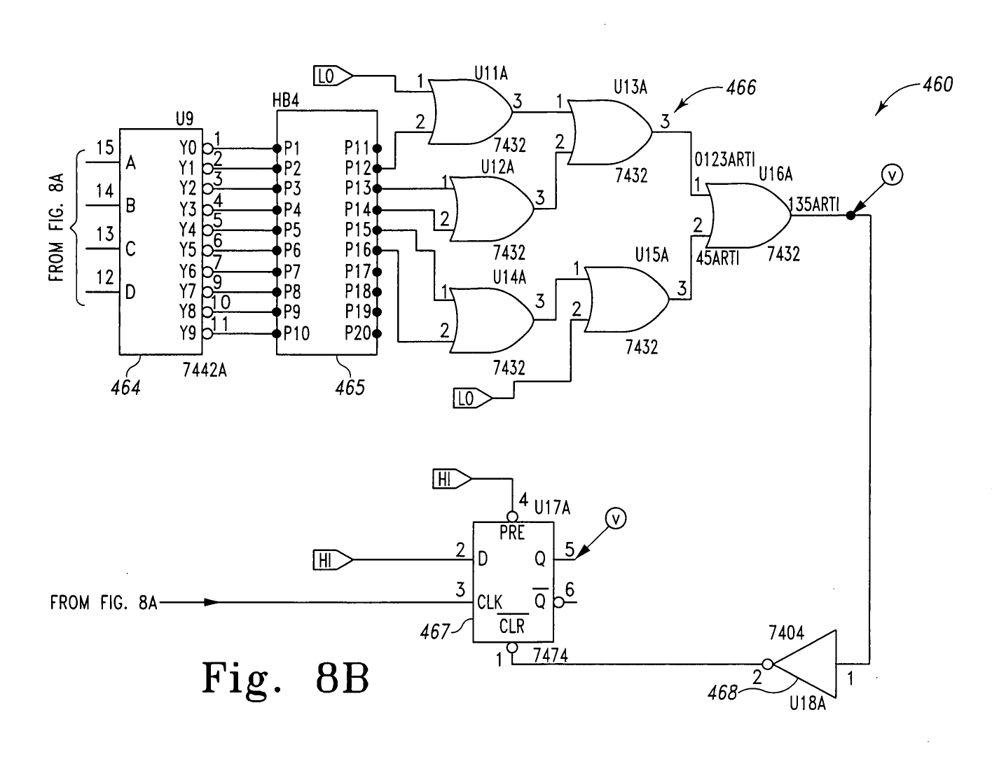

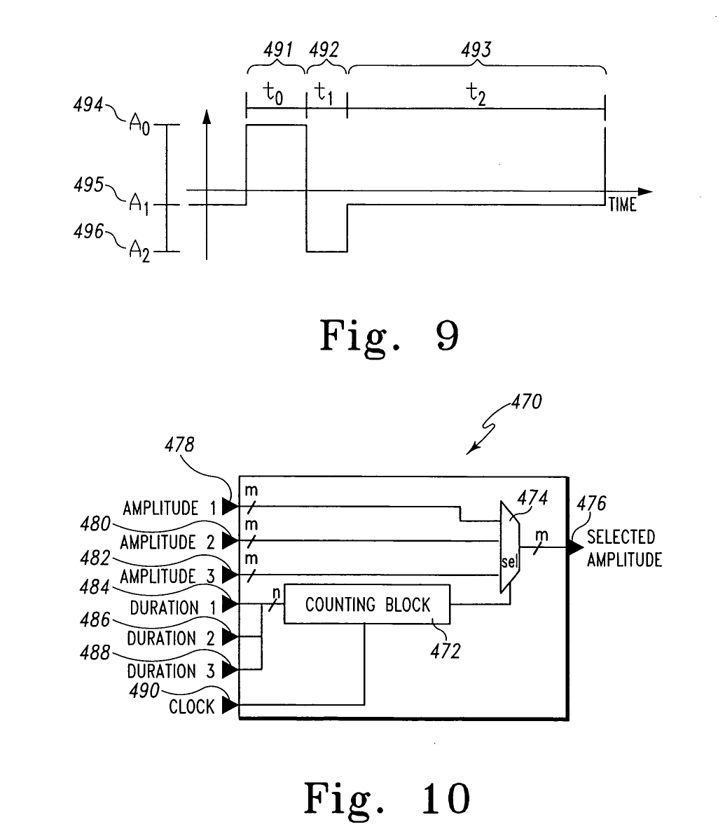

The stimulator module 420 may include a current source 422, a charge balance sensor 424, a pulse generator 426, an inductor 427, a field-to-current converter 428, and the charge storage device 429 described above in relation to FIG. 4A. The current source 422 is shown in more detail in FIG. 5. A biphasic embodiment 460 of the pulse generator 426 is shown in more detail in FIGS. 8A-B. A triphasic embodiment 470 of the pulse generator 426 is shown in more detail in FIG. 10. FIG. 9 shows a sample wave form generated by the triphasic embodiment 470 of the pulse generator 426.

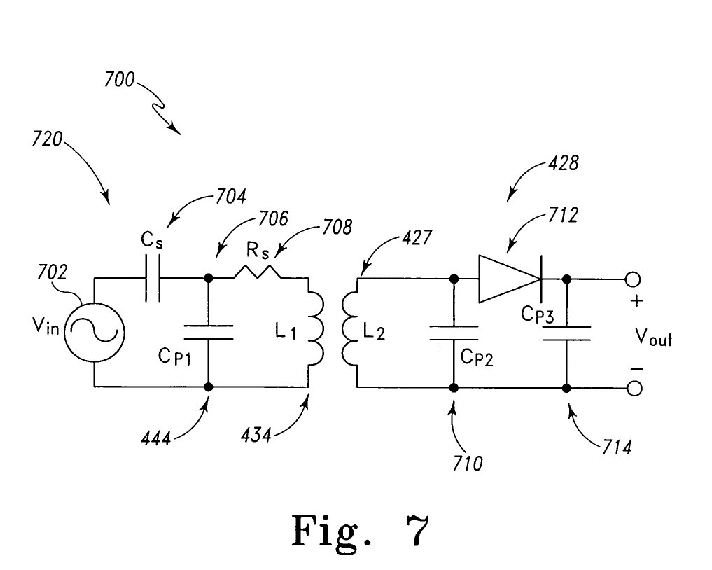

The inductors 427 and 434 and other power coupling components are shown in more detail in FIG. 7. The field-to-current converter 428 may be a radio frequency field-to-current converter. The stimulator module 420 may communicate via the wireless data module 410 with the external module 430 via antennas 418 and 432 respectively. The external module 430 may also include a subcutaneous charging device 444 for inductively charging the charge storage device 429 via field converter 428. The electrodes 440 comprises the Electrode Group A 442 and the Electrode Group B 444.

In operation, the wireless data module 410 receives power from stimulation module 420 that receives power from the external module 430, stores the power for a time in charge storage device 429, and uses the stored power to generate a field between the electrode Group A 442 and the Electrode Group B 444. The electromagnetic power coupling circuit 700, shown in FIG. 7, shows the field-to-current converter 428 in more detail. Additionally, the external portion 720 of the power coupling circuit 700 is also shown in FIG. 7. A voltage source 702 of the external portion 720 is coupled to an R-L-C circuit comprising first and second capacitors 704 and 706, a resistor 708, and an inductor 434. The external portion 720 generates an electromagnetic field, which may be induced into the inductor 427 of the field-to-current converter 428 when the inductors 434 and 427 are in proximity to one another. When that occurs, the inductor 427 provides an AC voltage to the simple rectifier circuit comprising first and second capacitors 710 and 714, and diode 712. In this manner, the field-to-current converter 428 may operate to transform coupled fields to direct current fields through charge-rectifying and/or signal conditioning. The field-to-current converter 428 may also regulate coupled power delivery for appropriate charging of the charge storage device 429.

Transcutaneous recharging of the charge storage device 429 can be accomplished using medically approved voltage sources such as the Quallion QL100E (weight 4 grams; capacity, 100 mAh; Operating Voltage 2.7-4.2 V; size 14.5 mm by 15.6 mm). The largest component of the circuit 400 determining its overall size is the size of the charge storage device 429. Thus, decreasing the size of the device by using a rechargeable unit for the charge storage device 429 may reduce the size of the circuit 400 to sixty percent (or a smaller percentage) of prior art devices. This decrease in size may simplify surgical implantation, and the time of implantation. Other medical issues, such as contact necrosis, also vary with the size of the circuit 400. The timing of recharging cycles will depend on the programmed stimulation parameters. However, charging could be accomplished at night while the patient is asleep, or for shorter periods during the day.

Since the circuit 400 may be located rather superficially in back musculature beneath the back skin, an additional pair of redundant recharging electrodes may be left in situ next to the circuit 400. These redundant recharging electrodes may be externalized simply by use of a local anesthetic and simple approach through the skin. Under special or unforeseen situations, the circuit 400 can be recharged directly by attachment of these two electrodes to a hardwired recharging unit.

Returning to FIG. 4, the charge storage device 429 may store power received from the field-to-current converter 428 up to its maximum capacity, which is monitored by the field-to-current converter 428 to avoid over-charging of the charge storage device 429. Upon reaching maximum capacity, the charge storage device 429 may contain enough power to power the circuit 400 for the appropriate length of time, and charging may cease.

As shown, for example, in FIG. 10 a triphasic pulse generator 470 includes a counter block 472, a multiplexer block 474 an output 476, a first amplitude input 478, a second amplitude input 480, a third amplitude input 482, a first duration input 484, a second duration input, 486, a third duration input 488 and a clock input. In one embodiment of the triphasic pulse generator 470 the counting block 472 comprises three counters and the data present at the amplitude inputs 478, 480, 482 and duration inputs 484, 486, 448 comprise six words of data stored in a form of memory (not shown). The three data words present at the duration inputs 484, 486, 488 are illustratively n bits long and represent the duration of each pulse, for example, duration t0 491, duration t1 492 and duration t2 493, as shown, in FIG. 9. The three data words present on the amplitude inputs 478, 480 , 482 are illustratively m bits long and represent the amplitudes of each pulse, for example, amplitude AO 494, amplitude Al 495 and amplitude A2 496, as shown, in FIG. 9. In the illustrated embodiment, the three counters in the counting block 472 reset with a value between a value between zero and 2n−1, where n is the number of bits contained in the counter. This number will represent a time until the counter rolls over. Upon rollover the counter send a flag initiating the next counter. The same operation applies for the second and third counter. While each counter is counting, a multiplexer 474 selects one of the three amplitudes stored in memory determined by the counter currently in operation. Power saving is accomplished by clock gating or reducing the number of counters needed to count the duration of the pulse.

In accordance with the present disclosure, the nerves in the central nervous system of a mammal are stimulated to regenerate by applying an oscillating electrical field to the central nervous system. The oscillating electrical field is a voltage potential stimulus which is first applied in one direction for a predetermined period of time, and then applied in the opposite direction for the predetermined period of time. In other words, the polarity of the voltage potential stimulus is reversed after each predetermined period of time. The predetermined period of time is selected to be less than the die back period of anodal facing axons, but long enough to stimulate growth of cathodal facing axons. This predetermined period will be termed the “polarity reversal period” of the oscillating electrical field. In one disclosed embodiment, this polarity reversal period is between about thirty seconds and about sixty minutes.

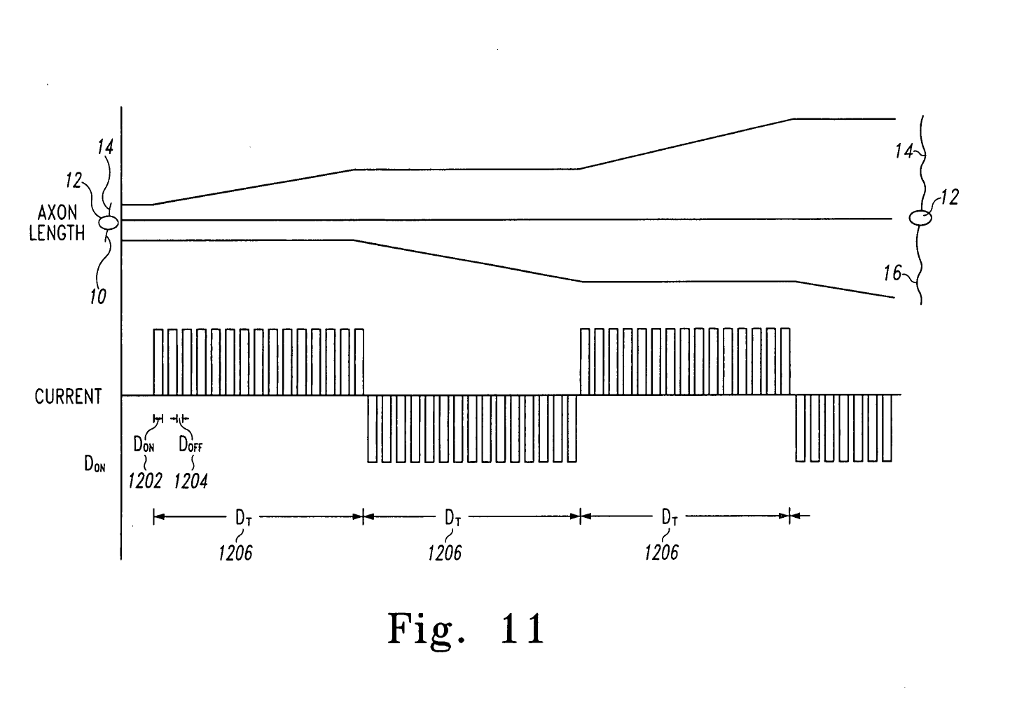

Circuit 400 when implemented with a biphasic pulse generator 460 (FIGS. 8A-B), triphasic pulse generator 470 (FIG. 10) or other multi-phasic pulse generator as the pulse generator 426 comprises a chopping circuit. The voltage potential difference and thus the electrical field between the electrode of the first and second implant 102, 104 is “chopped” or turn off for a short but fixed amount of time. For example, by setting jumper 620 to a 25% duty cycle and jumper 622 to a 50% duty cycle, the electrical field exhibits an on duty cycle Don 1202 of 75% (jumper 620 plus jumper 622) and off duty cycle Doff 1204 for 25% of the time, chopped once per minute producing a wave form as shown in FIG. 11. If this amount of time is small enough compared to the overall time, the nerve cell regeneration continues at the same rate as if the electrical field were held steady. However, chopping the electrical field in the manner illustrated increases battery life, or enables the battery to power other device functions while maintaining a lifespan sufficient for regeneration to be substantially completed. Additionally, punctuated, pulsatile or discontinuous oscillating electric fields are believed to work as well, if not, in some case when utilized to heal certain types of nerves, better than, constant oscillating electric fields. Thus, there is the expectation that the chopping circuit will generate a pulsatile electric field that may improve functional recovery as well as save battery life.

In one disclosed embodiment, where polarity reversal period DT 1206 of the oscillating electrical field is set to 10 minutes and the duty cycle of the electrical field is set to 75%, circuit 400 produces an output wave form as shown in FIG. 11. It is within the scope of the disclosure for the polarity reversal period to be between about thirty seconds and about sixty minutes. It is also within the scope of the disclosure for the polarity reversal period to be between a minimal clinically effective value to stimulate nerve regeneration in the cathode-facing axon and a value less than the beginning of the die-back period in the anode-facing axon. Clinically effective results can readily be obtained when the reversal period is set between ten and twenty minutes. Highly effective clinical results may be achieved with the duty cycle set to approximately fifteen minutes. It is also within the scope of the disclosure, though not preferred because regeneration of axons induced to die back through the area of die back will be required before therapeutic growth will be induced, for the polarity reversal period to exceed the beginning of the die back period but be less than the time for die back to proceed to the point of killing the nerve cell.

It is within the scope of the disclosure for the on duty cycle 1202 to be between 60% and 99%. Clinically effective results may be obtained in one embodiment when the on duty cycle 1202 is between 70% and 85%. Clinically effective results may be obtained in another embodiment when the on duty cycle 1202 is between 75% and 80%.

According to at least one embodiment of the present disclosure employing a pulsatile field, there may be an off cycle between each polarity reversal period, or there may be two or more consecutive polarity reversal periods followed by an off cycle.

As shown, for example, in FIGS. 8A-B, biphasic pulse generator 460 is implemented using a binary counter 461, a magnitude comparator 462, a buffer 463, a BCD-decimal decoder 464, a second buffer 465, a plurality of two input or gates 465, a NAND gate 466 and a D Flip Flop 467. Such off the shelf integrated circuits are available from many electronic device manufactures. Exemplary part numbers are shown in the drawings. The biphasic pulse generator 460 is configured to receive a plurality of high and low inputs and a fed back clock signal at the binary counter 461. Pulsed signals output by the binary counter 461 are input to the comparator 462 along with various hi and low inputs in the manner illustrated. The biphasic pulse generator 460 outputs a signal such as that shown for example in FIG. 11.

The charge storage device 429 provides power to the current source 422, the charge balance sensor 424, and the pulse generator 426. The pulse generator 426, shown in more detail in FIGS. 8A-B and/or FIG. 10, may generate a therapeutic waveform, as will understood by those of skill in the art of digital electronic design. For example, the pulse generator 426 may generate a pulsatile DC or intermittent DC waveform (as described above). The current source current source 422 may comprise a plurality of current sources 450, one illustrative embodiment which is shown in FIG. 5. The waveform generated by the pulse generator 426 may be provided to the plurality of current sources 450, as shown in FIG. 4, so as to generate a pulsatile DC or intermittent DC field of the desired nature between the electrode Group A 442 and the Electrode Group B 444.

Turning to FIG. 5, the current source 450 includes a biasing current source 451, first, second, and third field-effect transistors 452, 456, and 458, respectively, and an operational amplifier 459. The current source 450 receives a voltage waveform from the biphasic pulse generator 426 (which is represented as V_{DD} in FIG. 5), and provides a current I_{OUT} at transistors 458, which is provided to one of the electrodes of Electrode Group A 442 or Electrode Group B 444.

The wireless data module 410 may facilitate the treatment of individual patients by allowing the clinician to vary the therapy to adapt to the anatomical and/or physiological pathology of individual patients, which varies considerable after spinal cord injury. The capability of the clinician to interrogate the implanted circuit 400 and to change its stimulation parameters via the external module 430 may facilitate custom applications of the therapy. For example, excessive scar tissue may build up about electrodes tethered to the paravertebral musculature, and reduce the strength of the imposed field (reducing current flow by increasing interstitial resistivity). This is not ideal for the success of the therapy. For example, where a reduction in voltage between the Electrode Group A 442 or Electrode Group B 444 is detected by the charge balance sensor 424, two-way telemetry between the wireless data module 410 and the external module 430 allows for correction by increasing the voltage produced by the current source 422. For further example, where a drop or loss in voltage be detected in only one pair of the electrodes in the Electrode Group A 442 and Electrode Group B 444, a correction may be initiated by telemetry from the external module 430 instructing the wireless data module 410 to increase the delivery of adjacent pairs of electrodes. In essence, two-way telemetry provides for changes in stimulation parameters and the ability to correct and/or tailor the regenerative electrical stimulation.

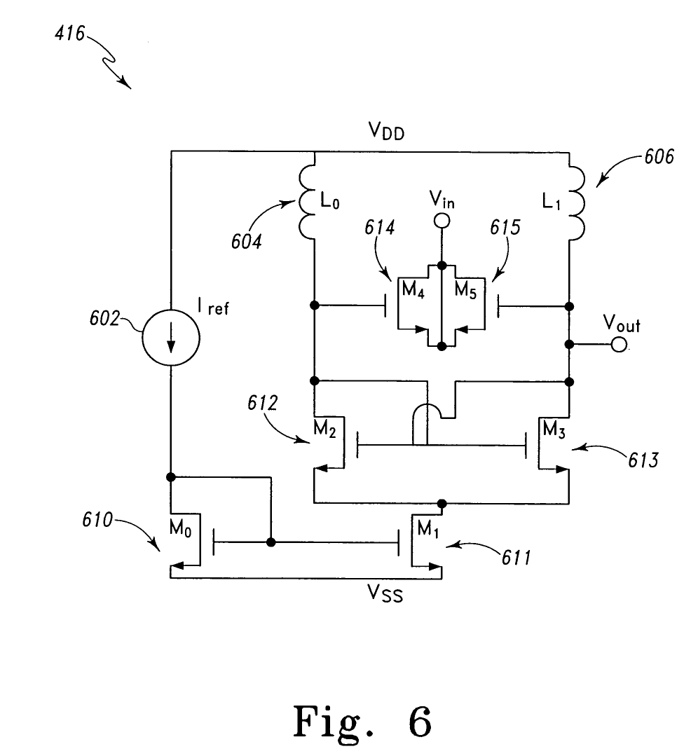

In operation, the transceiver 414 transmits and receives signals via the antenna 418, which signals may be radio frequency signals. The transceiver 414 is coupled to a voltage controlled oscillator 416 (shown in detail in FIG. 6), and a low-pass filter 412. As shown in FIG. 6, the voltage controlled oscillator 416 includes transistors 610-615, inductors 604 and 606, and a current source 602. The voltage controlled oscillator 416 receives a data signal at Vin, and generates an oscillated signal at Vout.

Returning to FIG. 4, the wireless data module 410 transmits data between the stimulator module 420 and the external module 430, thereby facilitating two-way telemetry. The voltage controlled oscillator 416 and external module 432 shown in FIGS. 4A and 4B are merely illustrative, and do are not intended to limit the claimed invention in any way.

While this invention has been described as having a preferred design, the present invention can be further modified within the scope and spirit of this disclosure. This application is therefore intended to cover any variations, uses, or adaptations of the invention using its general principles. For example, the methods disclosed herein and in the appended claims represent one possible sequence of performing the steps thereof. A practitioner of the present invention may determine in a particular implementation of the present invention that multiple steps of one or more of the disclosed methods may be combinable, or that a different sequence of steps may be employed to accomplish the same results. Each such implementation falls within the scope of the present invention as disclosed herein and in the appended claims. Furthermore, this application is intended to cover such departures from the present disclosure as come within known or customary practice in the art to which this invention pertains and which fall within the limits of the appended claims.

What is claimed is:

1. An apparatus for stimulating axon growth of the nerve cells in the spinal cord of mammals to stimulate regeneration of the nerve cells in the spinal cord, the apparatus comprising:
a variable current DC stimulus generator having first and second groups of electrodes, wherein one group of electrodes comprises at least three electrodes acting as a cathode of the generator, and the other group of electrodes comprises at least three electrodes acting as an anode of the generator;
polarity reversing circuitry configured to reverse the polarity of the DC stimulus each time a predetermined period of time elapses, wherein each time the polarity of the DC stimulus is reversed the electrodes which comprised the cathode before the polarity reversal comprises the anode after the polarity reversal and the electrodes which comprised the anode before the polarity reversal comprises the cathode after the polarity reversal; and
data transfer circuitry in communication with the DC stimulus generator, the data transfer circuitry operable to transmit signals to and from the DC stimulus generator.

2. The apparatus of claim 1, further comprising a DC stimulus controller that controls the duty cycle of the DC stimulus generator to provide an on-cycle wherein the generator provides a DC output and an off cycle wherein the generator does not provide a DC output, the duty cycle being generated during each polarity reversal.

3. The apparatus of claim 1, further comprising a DC stimulus controller that controls the amplitude of the DC stimulus generator to provide an on-cycle wherein the generator provides a DC output and an off cycle wherein the generator does not provide a DC output, the duty cycle being generated during each polarity reversal.

4. The apparatus of claim 1, further comprising a DC stimulus controller that controls the frequency of the DC stimulus generator to provide an on-cycle wherein the generator provides a DC output and an off cycle wherein the generator does not provide a DC output, the duty cycle being generated during each polarity reversal.

5. The apparatus of claim 1, wherein the variable current DC stimulus generator, the groups of electrodes, and the data transfer circuitry are each components configured to be implanted in the body of a patient suffering nerve cell damage.

6. The apparatus of claim 1, further comprising an external controller for controlling the output of the DC stimulus generator.

7. The apparatus of claim 6, wherein the external controller is communicatively coupled with the data transfer circuitry.

8. The apparatus of claim 6, wherein the external controller and the data transfer circuitry are capable of bi-directional communication.

9. The apparatus of claim 8, wherein the bi-directional communication is accomplished via radio frequency transmission.

10. The apparatus of claim 1, further comprising a rechargeable charge storage device coupled to the variable current DC stimulus generator.

11. The apparatus of claim 1, wherein the data transfer circuitry comprises at least one transceiver and at least one antenna.

12. The apparatus of claim 1, further comprising at least one sensor capable of monitoring the electrical environment surrounding the apparatus.

13. The apparatus of claim 12, wherein at least one electrode is configured as the at least one sensor.

14. The apparatus of claim 1, wherein the data transfer circuitry is capable of telemetering information about the electrical environment surrounding the apparatus to an external device.

15. The apparatus of claim 14, wherein the external device in response to the information about the electrical environment surrounding the apparatus generates configuration information and transmits the configuration information to the data transfer circuitry, the configuration information comprising parameters for controlling the output of the DC stimulus generator.

16. The apparatus of claim 1, further comprising at least one sensor capable of monitoring the electrical environment surrounding the apparatus.

17. The apparatus of claim 16, wherein at least one electrode is configured as the at least one sensor.

18. The apparatus of claim 1, wherein the data transfer circuitry is capable of telemetering information about the biological environment surrounding to an external device.

19. The apparatus of claim 18, wherein the external device in response to the information about the biological environment surrounding the apparatus generates configuration information and transmits the configuration information to the data transfer circuitry, the configuration information comprising parameters for controlling the output of the DC stimulus generator.

20. A nerve cell injury treatment system comprising:
a biocompatible container;
a wireless data module within the biocompatible container, the wireless data module comprising at least at least one transceiver, the wireless data module operable to transmit and receive data;
a stimulator module within the biocompatible container and electrically connected to the wireless data module, the stimulator module configured to reverse the polarity of a DC stimulus each time a predetermined period of time elapses;
an external module located outside the biocompatible container, the external module comprising data acquisition circuitry and data transmission circuitry, wherein the external module and the wireless data module are communicatively coupled via radio frequency communication, and wherein the external module in response to the data transmitted by the wireless data module is operable to generate and transmit configuration information to the wireless data module, the configuration information comprising parameters for controlling the stimulator module;
a first group of electrodes, each electrode of the first group of electrodes comprising a distal end and a proximal end and an electrically conductive material between the distal end and the proximal end, each proximal end being located within the biocompatible container and electrically connected to the stimulator module, and each distal end located outside the biocompatible container, each electrode of the first group of electrodes being responsive to the DC stimulus; and
a second group of electrodes, each electrode of the second group of electrodes comprising a distal end and a proximal end and an electrically conductive material between the distal end and the proximal end, each proximal end being located within the biocompatible container and electrically connected to the stimulator module, and each distal end located outside the biocompatible container, each electrode of the first group of electrodes being responsive to the DC stimulus;
wherein one group of electrodes acts as a cathode of the DC stimulus, and the other group of electrodes acts as an anode of the DC stimulus, and wherein each time the polarity of the DC stimulus is reversed the first electrode group which comprised the cathode before the polarity reversal comprises the anode after the polarity reversal and the first electrode group which comprised the anode before the polarity reversal comprises the cathode after the polarity reversal.
